(19) United States
(12) Patent Application Publication
(10) Pub. No.: US 2010/0215672 A1
(43) Pub. Date: Aug. 26, 2010

(54) Preparation process and utilization of Poncirus compositions having anti-allergic and antianaphylactic properties

(76) Inventor: Charles R. de Charleroy, JR., (US)

Correspondence Address: Charles R. de Charleroy, Jr., 304 Serrano Trail, Browns Mills, NJ, 08015-5116, US

(21) Appl. No.: 12/393,280

(22) Filed: Feb. 26, 2009

(51) Int. Cl. A61K 39/00; A61K 36/752; A61K 31/35; A61K 9/127; A61K 9/48; A61K 36/82

(52) U.S. Cl. 424/184.1; 424/736; 514/456; 424/450; 424/456; 424/729

(57) ABSTRACT

A composition which comprises extracts of a plant of the genus Poncirus. The composition effectively treats allergic conditions, atopic conditions, anaphylactic conditions, and autoimmune conditions.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Field of the Invention

The present invention relates to a novel composition, and particularly to a novel composition comprising an extract of the fruit and/or seeds of a plant of the genus Poncirus (may be abbreviated to Poncirus extract hereinafter).

2. Description of the Prior Art

Allergic diseases have frequently proven difficult to treat in many cases even with advances in modern medicine. Some, such as anaphylactic-type reactions, are life-threatening at every occurrence and are currently only treatable with medications that induce potentially dangerous side-effects, such as severe drowsiness or heart problems.

Alternative medical practice has sought to use natural remedies for allergies, which can have fewer side effects, but with little effect on the most severe forms of allergic reactions. Many plants do not possess effective compounds in enough quantity to be deemed useful for treating allergic diseases or must be combined with other ingredients.

Most plants used in natural remedies for allergies that have a demonstrated effect are tropical in nature, and thus have limited growing range and are difficult to grow in great enough quantities to serve the general public.

### REFERENCES

1. Park S H., et al., “Passive cutaneous anaphylaxis-inhibitory activity of flavanones from Citrus unshiu and Poncirus trifoliate,” Planta Med., 2005 January;71(1):24-7
2. Kim H M., et al., “Inhibition of immunoglobulin E production by Poncirus trifoliata fruit extract,” Journal of Ethnopharmacology, September;66(3):283-8
3. Lee Y M., et al., “Effects of Poncirus trifoliata on type I hypersensitivity reaction,” Am J Chin Med, 1997;25(1):51-6
4. Lee Y M., et al., “Antianaphylactic activity of Poncirus trifoliata fruit extract,” Journal of Ethnopharmacology, 1996 November; 54(2-3):77-84
5. The Japanese Pharmacopoeia, 13^{th} Edition, p. 34 (1996)

### BRIEF SUMMARY OF THE INVENTION

The present inventor analyzed plants used in Chinese folk medicine which had a long history of use and widely accepted non-toxicity, as well as having a high degree of adaptability and growth range large enough for the plants to be farmed extensively. Plants of the genus Poncirus, which originated in China and have naturalized into many countries since, were found to fit the criteria and have been used to treat allergies by native populations in China. The effectiveness of the raw fruit extract is quite potent, and a long history of use both medicinally and in jellied forms demonstrates that it is quite non-toxic. However, bitterness and low yield of extract remained problems for common use. A composition has been prepared which eliminates the bitterness while retaining the full effectiveness of the raw extract prepared by the usual methods. This extract can be administered orally and is used to treat and prevent diseases. Therefore, the present inventor has accomplished this invention.

### DESCRIPTION OF THE PREFERRED EMBODIMENT

The composition of the present invention contains a processed product of a plant of the genus Poncirus (designated as “Poncirus”, hereinafter). The processed product used in the present invention includes processed products which can be obtained by chemically and/or physically affecting plants and plant components of Poncirus, and the materials and methods are not restricted specifically. Primarily, the species used in the present invention is Poncirus trifoliata. Included in the treatments, both physical and chemical, used in the present invention are mincing, disrupting, pulverizing, grinding, extracting and/or fermenting the plants and parts of the plants. The processed products contain one or more of the flavonoids ponciretin, hesperetin, and naringenin, and may include one or both of the aglycone and flavone glycoside derivatives of the said flavonoids.

The plants of the genus Poncirus include naturally and artificially grown plants and their mutants which are obtainable by conventional breeding of the plants. As for the plant materials used as starting materials, the following may be used arbitrarily; the fruit, leaves, stems, rinds, and seeds, which are separated from the plant bodies. These plant materials can be used in fresh, frozen, dried, or dehydrated forms. In particular, the fresh fruits of Poncirus trifoliate can be used in the present invention.

The processed Poncirus product used in the present invention can be obtained by utilizing the starting materials and subjecting them to physical and/or chemical treatments used by the food and pharmaceutical industries. Conventional treatments including grinding, pulverizing, mincing, disruption, fermenting, pressing, and extracting alone or in combination with other techniques such as concentration, drying, and filtering can produce the present processed products.

To explain in more detail the production process of Poncirus extracts which are used as the processed products of Poncirus; the starting materials are treated with mincing, disruption, pulverization, etc., and the resulting mixtures are then treated with conventional extraction methods using solvents, which include water, aqueous organic solvents, polar organic solvents and hydrophobic organic solvents. In particular, water and aqueous organic solvents are preferably used as solvents for extraction. The aqueous organic solvents can include ethanol, methanol, propanol, acetone, etc. The organic solvents are used in extraction in 50 v/v % or lower of their aqueous sytems. The solvents can be adjusted to a desired pH level by buffering chemicals and systems. The solvents are added to the starting material in an amount of 0.1-20-folds by weight, and treated by stirring and heating methods for extraction. The resulting extracts are separated into liquid and residual materials by methods such as filtration, centrifugation, and decanting, followed by concentration of the liquid portion of the Poncirus extract to be used in the present invention. The liquids parts of repeated extractions of the residual materials can be pooled together. Different extraction solvents can be used in each extraction when applying at least twice extractions to the residual parts, followed by collection and concentration of the liquid parts into the Poncirus extract used in the present invention.

The extracts obtained thusly can be used as processed Poncirus products after use of appropriate purification treatments. The extracts contain secondary metabolites such as terpenoids, and also alkaloids, pigments, phenols, saccharides, proteins, amino acids, lipids and peptides. Desired compounds from these extracts can be purified by applying appropriate purification methods. Appropriate methods include filtration, centrifugation, solvent-based separations, concentration, crystallization, dialysis, sedimentation, adsorption chromatography, reverse-phase chromatography, hydrophobic chromatography, gel filtration chromatography, ion-exchange chromatography, and/or affinity chromatography.

The obtained processed Poncirus product obtained thusly can be used intact in the present invention and/or can be further concentrated, admixed with later described ingredients, or dried into solids, semi-solids, pastes, powders, liquids, suspensions, emulsions, etc. Conventional methods for concentration and drying used by the food and pharmaceutical industries can be used; vacuum concentration, vacuum drying, reverse-osmosis membrane concentration, membrane filtration, spray drying, and freeze drying.

The processed Poncirus product used in the present invention usually contains 0.1-10 w/w % flavone on a dry solid basis and may contain flavones derivatives. The solid content of the product can be calculated by using the water content obtained with the dry reduction test as described in The Japanese Pharmacopoeia, 13^{th} edition, pg 34 (1996).

In the present invention the favonoids hesperetin, naringenin and ponceritin and their aglycones and isoflavone derivatives, as well as enzyme-treated versions of these flavonoids can be arbitrarily used. The enzyme-treated flavonoids obtained through the action of de-glycosylating enzymes are preferred as they are most active physiologically for the immunologic effects claimed by the present invention.

The composition according to the present invention may include processed products of plants containing caffeine and/or amines. More specifically, processed products of the plant Camelia sinensis may be used. The processed products may be obtained through production methods not specifically restricted. The processed products may contain caffeine and/or an amine.

The processed products of caffeine- and/or amine-containing plants used in the present invention can be obtained by subjecting the plant or plant parts to physical and/or chemical treatments as are generally used in the food and pharmaceutical industries, such as mincing, crushing, grinding, pressing, pulverizing, fermenting, extracting, drying, concentration, and filtering.

The above processed products of a caffeine- and/or amine-containing plant can then be combined with the processed Poncirus products as mentioned above by mixing optimally in a ratio of between 0.01 to 1.00 of the caffeine- and/or amine-containing products with the processed Poncirus products, thusly obtaining the forms described in the current invention. The mixing method used is not specifically restricted.

The present compositions exert strong anti-allergic, anti-anaphylactic, and immunoenhancement effects. Since the inherent bitterness of Poncirus is improved and/or masked in the processed Poncirus products of the current invention, the present compositions can arbitrarily be used as orally administrable compositions which can promote and maintain health and can exert satisfactory therapeutic and preventative effects upon diseases.

The wording anti-allergic effects and anti-anaphylactic effects as referred to in the present invention include one or more of the following inhibitory activities toward allergies: (1) inhibition of histamine production, (2) inhibition of histamine release, (3) inhibition of IgE production, (4) inhibition of complement activation, (5) inhibition of T-cell antigen sensitization and inflammatory cytokine production by sensitized T-cells. The present compositions exert significant inhibitory effects on the production and release of histamine and the production of IgE.

The wording immunoenhancement effects as referred to in the present invention include one or more of the following activities for promotion, differentiation, and/or activation of immunocompetent cells of mammals and humans: (1) enhancement of natural killer cell activity against tumor cells and viruses, (2) enhancement of cytotoxic T-cells growth and lethal activity against tumor cells and virus-infected cells, (3) enhancement of the growth of T-helper cells.

As described foregoing, the present compositions possessing anti-allergic, anti-atopic, anti-anaphylactic, anti-asthmatic, and immunosuppressant effects can be used to inhibit allergic reactions, enhance immune systems, and stabilize immune-related conditions; and exert exceptional effects in the treatment and/or prevention of allergic diseases such as sinusitis, hayfever, asthma, anaphylaxis, atopic dermatitis, contact dermatitis, rheumatism, ulcerative colitis, alimentary allergy, and celiac sprue.

Forms of the present composition include those which are orally administrable products in general: juice beverage, soft drink, sports drink, chewing gum, jelly, jam, cream, gummy, alcohol, and cream.

Additionally, the present compositions include other types of compositions which contain one or more physiologically-acceptable pharmaceutical adjuvants, carriers, stabilizers, excipients, and dilutants, and agents. Examples of such stabilizers are saccharides such as glucose, lactose, maltose, trehalose, sucrose, sugar alcohols such as maltitol, sorbitol, xylitol, and mannitol; proteins such as serum albumin and gelatin; and buffers such as phosphoric acid, phosphate-buffered saline, carbonate, and citric acid salts. Examples of other biologically active substances include aspirin, ibuprofen, cyclosporine, vitamins A, B, C, D, E, and K, amino acids, calcium lactate, calcium glycerophosphate, potassium iodide, heparin, ginseng extract, gingko biloba extract, aloe extract, lactic acid bacterium, royal jelly, interferon, and optimally diphenhydramine.

The present compositions include pharmacologically acceptable forms in dosage units such that those containing processed Poncirus products are in an amount one to several folds of a dose by integers up to four fold or in an amount as measures of a dose down to 1/40, and have physically integrated forms for ease of administration. Examples of such forms are liquids, powders, tablets, granules, capsules, suppositories, external medicines, injections, and nebulae. The present compositions are orally and/or parenternally administrable to patients and exert satisfactory therapeutic and preventative effects on susceptible diseases independent of their administration routes. The compositions can be administered in forms such that allow administration by intradermal, subcutaneous, intramuscular, or intravenous routes, in a dose of between 0.1 mg to approximately 10 g/dose/adult, and preferably about 1 mg to 1 g/dose/adult of processed Poncirus products, depending upon the types and symptoms of the susceptive disease, 1-4 doses per day or 1-5 shots per week for one day to one year.

The following experiments 1 to 4 describe the embodiments of the function and taste of a primary composition of the aqueous extracts of the fruit of a plant in the genus Poncirus according to the present invention:

### Experiment 1

### Preparation of the Processed Products

### Experiment 1-1

### Poncirus Extract

The freshly obtained fully-ripened fruits of Poncirus trifoliata were allowed to rest in an open dish in a warm, dry location at a temperature of at least 25° C. for 3 days. 100 grams of fresh fruit pulp of Poncirus trifoliate were separated from the fruit by mashing with a hand-held metal mashing instrument and straining out the seeds. 30 ml of distilled water was added to the filtrate and the resulting slurry was pureed in a Hamilton Beach 10-Speed Blendmaster blender at highest speed for 1 minute. The mixture was strained again through filter paper to give a primary aqueous extract of between 40 and 50 ml.

The primary aqueous extract was placed in a wide glass bowl and reduced via vacuum evaporation in a conventional plastic vacuum chamber for approximately 6 hours, or until the volume was reduced to a final aqueous extract volume of between 15-20 ml.

### Experiment 1-2

### Camelia Sinensis Extract

Approximately 10 grams of dried Camelia sinensis leaves were placed in a Styrofoam vessel and boiled in 50 ml of distilled water in a General Electric Spacemaker microwave oven for 2 minutes, then allowed to soak for 2 minutes afterward. The resulting aqueous extract was reduced via vacuum evaporation in a conventional plastic vacuum chamber for approximately 6 hours, or until the volume was reduced to a final aqueous extract volume of between 15-20 ml.

### Experiment 2

### Anti-Allergy Action

Experiments previously conducted upon mice demonstrated effects upon IgE and histamine release which indicated Poncirus might have a therapeutic effect on human allergic diseases. Long-term use of Poncirus fruits and juices as a food and simple aqueous solutions by native human populations with no reported adverse effects indicates that for typical dosages, Poncirus extracts are expected to have no toxicity.

TABLE 1 Response of allergy symptoms. Note: Each dose comprised 10 ml of the aqueous extract mixed into 250 ml water or other beverage for oral administration. Table numerals in the ‘Allery Severity” column from 1-10 indicate symptom severity for that day, with 1 being the lowest severity and 10 being the highest severity. A Poncirus extract obtained by the method in Experiment 1-1 was tested upon 2 volunteers with seasonal allergies. The volunteers were given an allergy symptom assessment form and instructed to record their symptom severity for 21 days; 5 days before taking the Poncirus extract, 10 days during the administration of the extract, and then for 6 days after administration. Each volunteer was then given 10 doses of the Poncirus extract in orally-administrable liquid aliquots of 20 ml and instructed to take one aliquot per day for 10 days and record their personal assessment of allergy symptoms on a scale of 1 to 10;1 being very severe, 10 being no symptoms.

After the 21-day period, responses were analyzed and it was found that the volunteers reported a very significant reduction in their reported allergy symptoms during the period during which the Poncirus extract was administered. The effect also extended several days beyond the end of the administration period.

### Experiment 3

### Anti-Anaphylactic Effect

A Poncirus extract obtained by the method in Experiment 1-1 was tested upon 1 volunteer with slow-latency intestinal anaphylactic reactions to food-borne fungi with accompanying allergic dermatitis, and also nasal allergies to airborne fungi and fungal spores, a lesser pathology in the anaphylactic spectrum.

TABLE 2 Record of allergic symptoms/anaphylactic episodes during study period. Note: Each dose was equal to 10 ml of the Poncirus extract, either in pure form or diluted in approximately 250 ml of a beverage for oral administration.

The volunteer was given an allergy symptom assessment form and instructed to record symptom severity for 30 days during the autumn season, during which time the subject had historically the most frequent occurrence of anaphylactic reactions. This was done for two concurrent years. The study period consisted of 7 pre-administration days before administration of the Poncirus extract, 5 days during the administration of the extract, and then for 18 days after administration. The volunteer was then given 5 doses of the Poncirus extract in orally-administrable liquid aliquots of 20 ml and instructed to take one aliquot per day for 5 days and record a personal assessment of allergy symptoms on a scale of 1 to 10;1 being very severe, 10 being no symptoms.

The volunteer reported two episodes of anaphylaxis during the 7-day pre-administration reporting period of 2007 and three episodes of anaphylaxis during the 7-day pre-administration period of 2008. During the administration period, no episodes of anaphylactic or other allergic disease symptoms were reported. Also, for the 18-day period after the end of administration, no anaphylactic symptoms were reported and nasal allergic symptoms remained limited, indicating a possible long-term effect.

### Experiment 4

### Taste of Composition

A Poncirus extract obtained by the method in Experiment 1-1 was divided into aliquots of approximately 5 ml and sampled by 3 volunteers in the following ways: pure, undiluted extract; diluted in 125 ml water, diluted in 125 ml citrus juices, diluted in 125 ml iced tea beverage.

The four samples were evaluated on the basis of palatability grades: (1) freely taken orally, (2) freely taken orally, but possessing bitterness and harsh flavor, (3) unwillingly taken orally due to bitterness and harsh flavor.

TABLE 3 Palatability responses of volunteers to Poncirus extract oral compositions. Note: The symbols “UE”, “WD”, “CD”, and “TD” mean undiluted extract, water dilution, citrus dilution, and tea dilution, respectively.

As shown in Table 3, the taste of the undiluted Poncirus extract is considered unpalatable for willing consumption. The dilution in water improves the palatability, but still retains some of the undesirable aspects of the taste. The citrus juice and iced tea beverage dilutions greatly diminished the unpleasant aspects of the taste and gave indication that flavorant additives in addition to dilution successfully increase the palatability of the Poncirus extract and lead to compositions that can be successfully administered orally.

### Example Compositions

### EXAMPLE 1

### Liquid Formulation

Twenty ml of the concentrated aqueous Poncirus extract were combined with 20 ml ethanol, 10 ml high fructose corn syrup, 1 gram of citric acid, and 5 ml of polyethylene glycol in a glass beaker and mixed with a stainless steel spoon to create a syrupy liquid formulation. When taken orally, the product exerts its anti-allergic activity.

### EXAMPLE 2

### Powdery Composition

To 15 ml of the concentrated Poncirus extract obtained by the method in Eperiment 1-1, 60 g of alpha, beta-TREHALOSE, a trehalose product produced by Sigma-Aldrich, Co., was added and the mixture was treated by mixing in a glass beaker with a small plastic mixing spoon until homogenized. The mixture was then spread thinly upon aluminum foil and dried in vacuo at room temperature for 4 hours by a public laboratory vacuum source and a two-piece plastic vacuum chamber. A ceramic mortar and pestle was then used to pulverize the dried mixture into a powder containing the Poncirus extract.

To 15 ml of the concentrated Camelia sinensis extract obtained by the method in Eperiment 1-2, 60 g of alpha, beta-TREHALOSE, a trehalose product produced by Sigma-Aldrich, Co., was added and the mixture was treated by mixing in a glass beaker with a stainless steel mixing spoon until homogenized. The mixture was then spread thinly upon aluminum foil and dried in vacuo at room temperature for 4 hours by a public laboratory vacuum source and a two-piece plastic vacuum chamber. A ceramic mortar and pestle was then used to pulverize the dried mixture into a powder containing Camelia sinensis extract.

Fifty grams of the above powdery Poncirus extract and 10 g of a powdered Camelia sinensis extract were mixed in a glass beaker with a stainless steel mixing spoon to obtain a composition according to the present invention.

### EXAMPLE 3

### Iced Tea Beverage

To 250 ml of a cold beverage made from a solution obtained by steeping shredded Camelia sinensis leaves in water for 3 hours in a clear plastic container exposed to warm direct sunlight and straining out the leaves after the allotted time, 4 tsp of sucrose, and 20 ml of the concentrated Poncirus extract obtained from Experiment 1-1 were added to produce an iced tea beverage containing the present composition.

The product has acceptable levels of sweetness and bitterness, with a satisfactory flavor. The product has strong anti-allergic and immunoenhancement activities that function cooperatively and do not inhibit one another; thus it imparts physical activities to living bodies upon administration. The product effectively refreshes and strengthens bodies after periods of overwork, excessive physical exercise, and undernourishment, and promotes therapeutic effects.

### EXAMPLE 4

### Milky Composition

Twenty ml of concentrated Poncirus extract obtained by the method in Experiment 1-1, and 40 g of alpha, beta-TREHALOSE, a trehalose product produced by Sigma-Aldrich, Co., were homogenized. To the homogenized mixture, 5 g polyoxyethylene sorbitol tetraoleate, 5 g glycerol monostearate, 5 g isopropyl alcohol, and 10 g sesame oil with 1 g vitamin E were added. The mixture was conventionally dissolved and mized with 25 g 1,3-butylene glycol, 1 g carboxyvinyl polymer, and 750 ml distilled water, and emulsified in a homogenizer into a milky composition of the present invention. Then applied to skin, the product exerts its anti-allergic activity and can be used to treat/prevent allergic dermatitis and atopy.

### EXAMPLE 6

### Tablet Composition

A powdery composition obtained by the method in Example 2 was mixed with sucrose, corn starch, mineral oil, sodium pyrophosphate, and calcium carbonate, and the mixture was made into tablets using a 20 R punch to obtain tablets of approximately 12 mm in diameter. The product exerts satisfactory anti-allergic activity and imparts physical activity when administered to living bodies. The product is extremely low in toxicity. The present composition possessing these satisfactory properties can used in the food and pharmaceutical industries to maintain and promote health, and to treat or prevent susceptive diseases such as allergic diseases and anaphylactic diseases.

1. A composition comprising extracts of the fruit of a plant of the genus Poncirus comprising active formulants possessing anti-allergic, anti-atopic, anti-anaphylactic, anti-asthmatic, and immunosuppressant effects; wherein said extracts are aqueous and/or ethanol-based in nature and contain an amount of 0.01-10-folds of said extracts of the fruit of said plant of the genus Poncirus.

2. The composition of claim 1, wherein said extract is a pressed, squeezed, minced, disrupted, and/or extracted fruit and/or seeds of a plant of the genus Poncirus.

3. The composition of claim 1, which contains 0.01-20 w/w % of the extracts of a plant of the genus Poncirus.

4. The composition of claim 1, wherein the preferred embodiment of the extracts of a plant from the genus Poncirus is obtained from the species Poncirus trifoliata.

5. Formulations of the composition of claim 1 possessing selective immunoglobulin E release and production-inhibiting effects.

6. Formulations of the composition of claim 1 possessing histamine release-inhibiting effects.

7. Formulations of the composition of claim 1 possessing mast cell activity-inhibiting effects.

8. Formulations of the composition of claim 1 which may be used to treat and/or prevent an allergic condition including but not limited to nasal rhinitis, atopic asthma, seasonal allergies, intestinal anaphylaxis, respiratory anaphylaxic, and allergic dermatitis.

9. Formulations of the composition of claim 1 which may be used to treat and/or prevent symptoms of an allergic condition including but not limited to nasal rhinitis, atopic asthma, seasonal allergies, intestinal anaphylaxis, respiratory anaphylaxic, and allergic dermatitis.

10. Formulations of the composition of claim 1 possessing selective immunosuppressant activities.

11. Concentrated flavonoid/isoflavone formulations of the composition of claim 1 possessing selective immunoglobulin E release and production-inhibiting effects.

12. Concentrated flavonoid/isoflavone formulations of the composition of claim 1 possessing histamine release-inhibiting effects.

13. Concentrated flavonoid/isoflavone formulations of the composition of claim 1 possessing mast cell activity-inhibiting effects.

14. Concentrated flavonoid/isoflavone formulations of the composition of claim 1 which may be used to treat and/or prevent an allergic condition including but not limited to nasal rhinitis, atopic asthma, seasonal allergies, intestinal anaphylaxis, respiratory anaphylaxic, and allergic dermatitis.

15. Concentrated flavonoid/isoflavone formulations of the composition of claim 1 which may be used to treat and/or prevent symptoms of an allergic condition including but not limited to nasal rhinitis, atopic asthma, seasonal allergies, intestinal anaphylaxis, respiratory anaphylaxic, and allergic dermatitis.

16. Concentrated flavonoid/isoflavone formulations of the composition of claim 1 possessing selective immunosuppressant activities.

17. Pharmaceutical forms of the composition of claim 1 including but not limited too liquids, encapsulated gels, powders, solid tablets, liposomal formulations, semi-solid, paste, or suspension.

18. A method for producing the composition of claim 1.

19. A method for the treatment of allergic conditions with the composition of claim 1.

20. A method for the treatment of immunologic conditions with the composition of claim 1.

21. A method for the treatment of immunogenic conditions with the composition of claim 1.

22. Food products containing the composition of claim 1.

23. Beverages containing the composition of claim 1.

24. A formulation of the composition of claim 1 that includes extracts of a plant in the genus Camelia.
